(19) United States
(12) Patent Application Publication
(10) Pub. No.: US 2009/0036785 A1
(43) Pub. Date: Feb. 5, 2009

(54) BLOOD PRESSURE MONITOR

(76) Inventor: Per Danielsson, Ljungby (SE)

Correspondence Address: BROOKS KUSHMAN P.C., 1000 TOWN CENTER, TWENTY-SECOND FLOOR, SOUTHFIELD, MI, 48075, US

(21) Appl. No.: 12/278,040

(22) PCT Filed: Feb. 1, 2007
(86) PCT No.: PCT/SE07/00091

§ 371 Date: Aug. 1, 2008

(30) Foreign Application Priority Data
Feb. 1, 2006 (SE) 0600202-6

(51) Int. Cl. A61B 5/02

(52) U.S. Cl. 600/490

(57) ABSTRACT

Auto-focusing blood pressure monitor intended for “difficult” arms in order to make accurate and reliable measurements both at a normal arm and an arm with unusual formations, on which it has up to now been difficult to measure the blood pressure. The cuff has a cover and a bladder positioned therein. The bladder has two longitudinal chambers. A reinforcement extends along a valley between the chambers and operates as a tension ring, which maintains the division of the bladder in two chambers also at high internal pressure in the bladder. During application to a “difficult” arm, the reinforcement will be displaced towards the portion of the arm in which the problems are smallest while maintaining the division into two elliptical chambers. The elliptical chambers focus the pressure at the artery positioned there below so that a reliable measurement can be obtained.

### AREA OF INVENTION

The present invention relates to a blood pressure monitor comprising two inflatable cushions arranged in parallel.

### PRIOR ART

WO 88/00448, with the same inventor as in the present invention, discloses a blood pressure monitor with two elliptically shaped and parallel, inflatable cushions, enabling more accurate and reliable measurements than previously known technique.

Further demands on accurate blood pressure measurements prevail, especially with regard to the measurement of blood pressure at patients with difficult arms, strong muscles and unusual formations, as well as measuring blood pressure at patients with normal arms. Thus, there is a demand for a blood pressure monitor, which is universally usable.

If the blood pressure measurement is unreliable, it may be required to complement the blood pressure measurement with invasive measurement of the blood pressure directly in the artery, which may result in large costs and unnecessary suffering for the patient.

### DISCLOSURE OF THE INVENTION

An object of the invention is to provide a blood pressure monitor which is constructed and developed for making possible accurate measurements on both a normal arm and an arm with unusual formations, in which situation it has up to now been difficult to measure blood pressure.

Another object is to provide a blood pressure monitor, which is auto-convergent and finds the best place for measuring the blood pressure within certain limits adjacent the arrangement place of the blood pressure monitor.

In an aspect, there is provided a blood pressure monitor comprising a cover containing an inflatable bladder. Moreover, the blood pressure monitor comprises a reinforcement adapted approximately at the middle of the bladder and essentially along the length of the bladder. The reinforcement may comprise a fibre string arranged in a longitudinal pocket in the cover. Alternatively, the reinforcement can be a thickening at the middle of the bladder and along the entire length thereof. The reinforcement can be made of the same material as the bladder and integrally with the bladder. Alternatively, the reinforcement can be arranged along only a portion of the cover corresponding to the length of the bladder.

According to an embodiment, the reinforcement can be arranged to operate as a tension ring separating the bladder into two longitudinal chambers. These chambers may communicate with each other.

According to another embodiment, the reinforcement is cylindrical or rectangular. Moreover, the bladder may be preformed in two essentially elliptical chambers in which said reinforcement is positioned between the chambers.

### BRIEF DESCRIPTION OF THE DRAWINGS

Further objects, features and advantages of the invention will become evident from the following detailed description of embodiments of the invention with reference to the appended drawings, in which:

FIG. 1 is a perspective view of a bladder comprised in an embodiment of the blood pressure monitor;

FIG. 2 is a perspective view of a cover, in which the bladder according to FIG. 1 is arranged;

FIG. 3 is a cross-sectional view showing the blood pressure monitor arranged on an arm;

FIG. 4 is a cross-sectional view similar to FIG. 3, wherein the blood pressure monitor is automatically adjusted in dependence of muscles etc. in the arm;

FIG. 5 is a cross-sectional view of another embodiment of the blood pressure monitor;

FIG. 6 is a cross-sectional view of a further embodiment of the blood pressure monitor; and

FIGS. 7, 8 and 9 are partial cross-sectional views showing different manners of forming a reinforcement.

### DETAILED DESCRIPTION OF EMBODIMENTS

A conventional blood pressure cuff or blood pressure monitor comprises a cover of a flexible but relatively inelastic material, such as a woven material. A bladder of an elastic material is arranged inside the cover. The bladder is inflatable via a connection hose.

The cuff is intended to be applied on an arm of a patient, which is to be examined. The cover circumvents the arm and is usually attached with some form of Velcro-fasteners. The cover can be applied so that it is fixed with a suitable small pressure. Then, the bladder is inflated with pressurized air thereby squeezing an artery of the arm so that the blood flow there through is stopped. The pressure in the bladder is then slowly released and the pressure is read when the so-called Korotkoff sounds arise and disappear.

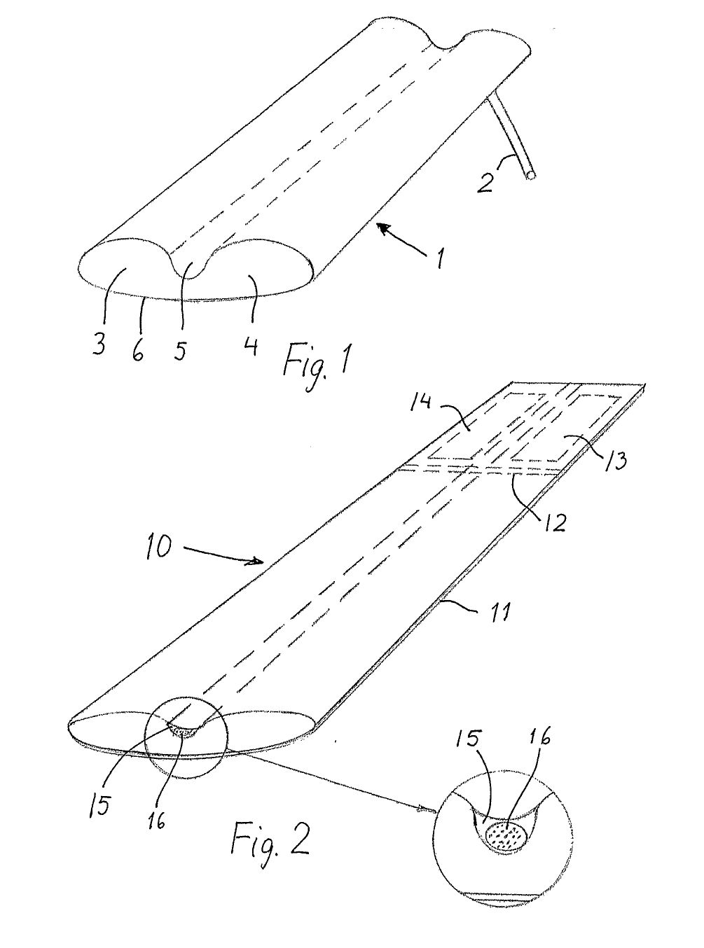

FIG. 1 shows a bladder, which is included in a blood pressure cuff according to an embodiment. The bladder is made of an elastic material, such as latex, synthetic or natural, or any elastomeric material, such as polyurethane.

The bladder 1 is closed as a balloon and has an inlet air hose 2. The bladder can be inflated by pumping in fluid, such as air or any other gas through the hose 2. The bladder is shown in FIG. 1 in a slightly inflated position.

The bladder is preformed with two longitudinal chambers 3, 4. A valley 5 between the chambers 3, 4 is positioned close to a backside 6 of the bladder. The chambers 4, 5 have a suitable length, which is longer than the length of the circumference of the arm of the patient to be examined.

As appears from FIG. 1, each chamber 3, 4 has a generally elliptic shape in its starting or initial position. When the pressure increases in the chambers 3, 4, the valley 5 will move longer away from the backside 6. Finally, the bladder becomes more or less balloon-shaped if it is inflated in a free condition. When the air is released again from the bladder, it automatically retains the preformed shape as shown in FIG. 1.

As appears from FIG. 1, the chambers 3 and 4 communicate freely so that they all the time has the same pressure. This is different from the blood pressure cuff as is disclosed in WO 88/00448, in which the chambers are separate and communicate via a hose connection.

The bladder is inserted in a cover during use, as shown in FIG. 2. The cover 10 consists of an elongated tube 11, over at least a part of the length. Thus, the tube forms a space, which is so dimensioned that the bladder fits therein more or less completely. The tube can be terminated by a seam 12 so that the space in which the bladder is positioned is delimited in the longitudinal direction. When the bladder has been inserted, the insertion end of the space is closed or sewn together, so that the bladder is not visible from the outside and is completely surrounded by the cover.

The cover 10 is made of a strong woven material, which entails a certain rigidity to the cuff. The cover has several attachment members, for example of the type Velcro fasteners 13, 14 at several places of the cover. By means of the fasteners, the cuff is attached to the arm in a manner not shown.

In the embodiment shown in FIGS. 1 and 2, the cover is provided with a longitudinal pocket 15 at the middle of the side, which is intended to abut the arm. In the pocket, there is arranged a reinforcement in the form of a fibre string 16, for example of carbon fibre material. The fibre string 16 extends along at least the entire length of the bladder. In FIG. 2 there is shown that the fibre string extends along the entire length of the cover. The fibre string 16 will be positioned in the valley 5 between the chambers 3, 4 when the bladder is inserted in the cover.

In FIG. 8, the pocket 15 and the fibre string 16 are adapted inside the cover, but the pocket can as well be attached or sewn at the outside, see FIG. 9. Another alternative embodiment is that the fibre string 16 is integrated with the pocket 15 by folding the pocket several times so that the folded material forms a reinforcement having the effect of a fibre string. The pocket does not need to be arrange by a separated material portion, but can be achieved by folding the cover material, see FIG. 7.

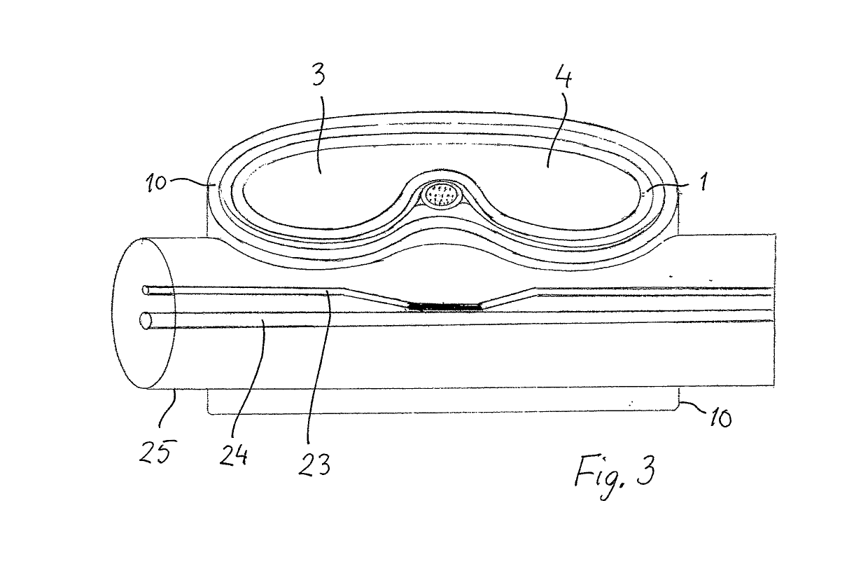

FIG. 3 shows the blood pressure cuff according to FIG. 1 and FIG. 2 applied on an arm. As appears from the figures, the upper side of FIG. 1 and FIG. 2 is the side, which is turned inwards towards the arm. Because the material of the cover is flexible, the cuff can be arranged around the arm and be attached by the Velcro-fasteners. When the cuff is arranged around the arm, its outer circumference cannot increase, since the cover material essentially is non-elastic.

A source of pressurized air is connected to the inlet 2 of the bladder and air is pumped into the bladder. This can take place with a hand pump or with a motor-driven pump or from a pressure vessel. The pressure in the bladder is measured continuously, for example with a manometer, such as a mercury manometer or an electronic pressure meter connected to the inlet 2. The pressure in the bladder is increased until it exceeds the arterial pressure. The artery in the arm is depressed, squeezed or strangled and no blood can pass. The pressure is relieved successively and the systolic pressure is read as the pressure when pulse sounds can be heard downstream of the cuff, for example with a stethoscope. The diastolic pressure is measured as the pressure when the pulse sound disappears again. The measurement can as well take place with an electronic meter, which listens after pulse sounds in the pressure signal of the bladder.

As appears from FIG. 3, the two elliptic chambers 3, 4 forms a force field, which focuses on a short measurement length of an artery, as described in WO 88/00448. In order that such a focusing effect should exist, it is essential that the chambers 3, 4 maintain their elliptic shape. This takes place by means of the reinforcement created by the fibre string 16. This fibre string is flexible but non-elastic and thus essentially non-elongateable or non-shortenable. However, it can be bent into a ring-shape, when the cuff is applied to the arm of a patient. The fibre string 16 is arranged around the arm and more or less clamped together with the cover. This means that the fibre string cannot be shortened when the bladder is successively inflated. Thus, the fibre string spans out the bladder at the middle and prevents the valley 5 from distancing itself substantially from the bottom 6. The fibre string operates like a tension ring, since it essentially cannot be shortened. Thus, the elliptic form of the chambers 3, 4 is maintained and the chambers can operate for focusing the force field.

The focused force field results in that the artery 23 there below is squeezed towards a tissue 24 positioned there below, such as a bone in the arm 25, along a relatively short squeeze length in the size of 20 to 40 mm, even if the cuff and the cover has a width of about 100 to 120 mm, i.e. the squeeze length is about one fifth to one third of the width of the cuff. In this way, an accurate measurement of the blood pressure is obtained as described in WO 88/00448. However, the measurements will be more accurate in the embodiment shown in FIG. 3, since the chambers 3, 4 are directly connected to each other, so that always the same pressure prevails in these two portions of the bladder.

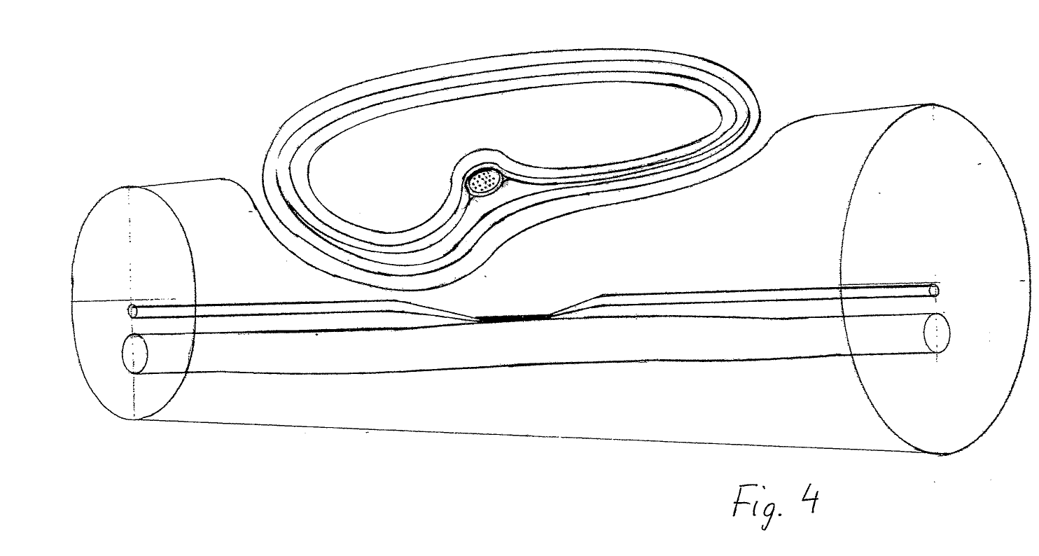

The embodiment shown in FIG. 3 of a blood pressure cuff is auto-convergent in such a manner that the cuff to a certain extent adapts itself to the structure of the arm there below, such as appears more closely from FIG. 4. If a portion of the arm, for example the right portion of FIG. 4, is comprised of a stiffer material, such as strong muscles, it will relax less than the portions of the arm positioned below the left portion of the cuff. It is then more favourable to measure the pressure closer to the left, softer portion of the arm, since the cuff then will come closer to the artery and can perform a more direct pressure on the artery. Since the bladder comprises two portions or elliptic chambers with the reinforcement 16 positioned there between, the left part of the cuff will sink deeper into the arm and the left portion of the bladder will become larger. Then, the middle of the cover will be displaced towards the left side, as clearly appears from FIG. 4. The reinforcement 16 will still divide the bladder into two chambers 3, 4 but with the valley 5 displaced somewhat to the left in FIG. 4. Since the reinforcement 16 cannot be shortened, is will operate as a tension ring and ensure that there is still formed two elliptic bladders with two force fields, now, however, displace towards the left side. The muscle at the right side will not prevent the measurement of the blood pressure, since the measurement point now automatically has been displaced to the left. Thus, the denomination “auto-convergent” blood pressure cuff.

The auto-convergent operation has been shown to be of extraordinary importance, resulting in that also patients with “difficult” arms can be examined. The auto-convergence takes place only to a certain extent limited by the width of the cuff and how much the middle point of the cover is displaced sideways. The displacement has in the practice shown to be up to 10 to 15 mm, which is completely sufficient in most cases.

The blood pressure cuff can, however, also with advantage be used at “normal” patients. It will then be of less importance if the cuff is put in an optimal position, since the cuff auto-converges itself to the right position. This makes it possible to the user to obtain a safer and more accurate result. In a stressful environment at an emergency department, the nurse can apply the cuff faster and obtain reliable blood pressure measurements even if it, of different reasons, is not possible to attach the cuff in an optimal position, for example due to an injury. It is also possible for the patient himself to apply the cuff and obtain consistent results, for example during use of an automatic meter.

In order to achieve the auto-convergence, the bladder needs to move in a certain extent in relation to the cover. This can be facilitated by providing the inner side of the cover with a wax-like layer, which makes easier such movement. Such a wax layer can be arranged on only one side of the cover turned upwards in FIG. 2 and thus towards the arm of the patient during use. It is also suitable that the bladder cannot substantially move in relation to the cover at the side facing downwards in FIG. 2. Thus, the cover may lack a wax layer at the inside and be rugged or in other manners have larger friction against the bladder. The bladder may as well be treated in the same way so that it has high friction towards the side facing downwards in FIG. 2 but low friction towards the side facing upwards in FIG. 2. Other materials than wax can be used, such as talk, in order to form a means for reduced friction between the bladder and the inside of the cover.

Another manner to achieve the described operation appears from FIG. 6. In this embodiment, the cover is provided with a thin cloth layer 21 between the bladder and the cover at the side facing towards the arm. The cloth is a flour cloth or a cocoon fabric and permits movement between the bladder and the cover.

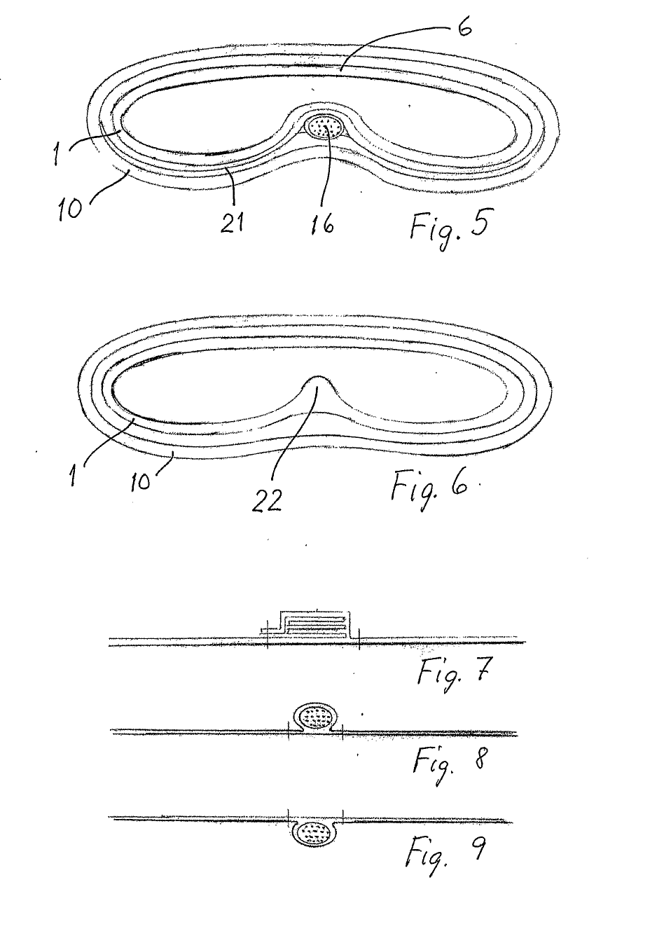

From FIG. 5, there appears also that the fibre string 16 can be a fibre string with cylindrical cross-section, for example a solid fibre string of nylon, carbon fibre or another man-made material. The fibre string 16 can also comprise several, for example ten or twenty, parallel cylindrical threads.

Another embodiment of the cuff is shown in FIG. 6. In this embodiment, the reinforcement 22 is arranged on the bladder and comprises an integral portion of the bladder. The reinforcement can be of the same material as the bladder and is then partly elastic, since the bladder is elastic. However, the reinforcement is much thicker than the rest of the bladder and operates thus as a tension ring as described above. Alternatively, the reinforcement can be of another, stiffer material and be attached to the bladder by means of a suitable adhesive, vulcanisation or welding, etc.

The reinforcement does not normally reach up to the artery and thus forms no strangling action of its own. However, in one embodiment, the reinforcement is attached to the outside of the cover and will partly aid in strangling the artery, which in certain cases can lead to better measurement results.

Such an arrangement appears from FIG. 9. The reinforcement may comprise a fibre string of the same material as the bladder, i.e. with a certain elasticity. The reinforcement can have a rectangular cross-section. Since the reinforcement is partly elastic, it can be compressed somewhat so that it will compress the arm and help to press the artery. At the same time, it fulfils its operation as a tension ring due to the fact that it has a relatively large height.

Herein above have been described embodiments of the invention in order for a skilled person to perform the invention. However, the invention is not limited to the described embodiments, features or constructions. The different features can be combined in other manners than those shown on the drawings. The invention is only limited by the appended patent claims.

1. A blood pressure monitor including a cover enclosing an inflatable bladder, comprising
a reinforcement adapted approximately at the middle of the bladder and essentially along the length of the bladder.

2. Blood pressure monitor according to claim 1, wherein the reinforcement comprises a fibre string arranged in a longitudinal pocket in the cover.

3. Blood pressure monitor according to claim 1, wherein the reinforcement comprises a thickening of the bladder adjacent its middle and along its entire length.

4. Blood pressure monitor according to claim 1, wherein the reinforcement is made of the same material as the bladder and integrally with the bladder.

5. Blood pressure monitor according to claim 2, wherein the reinforcement comprises a fold of the material of the cover.

6. Blood pressure monitor according to claim 2, wherein the reinforcement is arranged along only a portion of the cover, which corresponds to the length of the bladder.

7. Blood pressure monitor according to claim 1, wherein the reinforcement is arranged to operate as a tension ring, which separates the bladder into two longitudinal chambers.

8. Blood pressure monitor according to claim 7, wherein the chambers communicate directly with each other.

9. Blood pressure monitor according to claim 1, wherein the reinforcement is cylindrical or rectangular.

10. Blood pressure monitor according to claim 1, wherein the bladder is preformed into two essentially elliptic chambers wherein said reinforcement is positioned between the chambers.
